(19) United States
(12) Patent Application Publication
(10) Pub. No.: US 2003/0152983 A1
(43) Pub. Date: Aug. 14, 2003

(54) Desaturase

(75) Inventors: Johnathan A. Napier, Bristol (GB); Louise Michaelson, Bristol (GB); Keith Stobart, Bristol (GB)

Correspondence Address: BANNER & WITCOFF, 1001 G STREET N W, SUITE 1100, WASHINGTON, DC, 20001, US

(73) Assignee: University of Bristol, Bristol (GB)

(21) Appl. No.: 10/340,779

(22) Filed: Jan. 13, 2003

Related U.S. Application Data
(63) Continuation of application No. 09/582,034, filed on Dec. 19, 2000.
Application No. 09/582,034 is a 371 of international application No. PCT/GB98/03895, filed on Dec. 23, 1998.

(30) Foreign Application Priority Data
Dec. 23, 1997 (GB) 9727256.1
Jun. 29, 1998 (GB) 9814034.6

(51) Int. Cl. C12Q 1/68; C07H 21/04; C12N 9/04; C12N 1/18; C12N 15/82; A01H 1/00

(52) U.S. Cl. 435/6; 435/69.1; 435/190; 435/320.1; 435/325; 536/23.2; 435/254.2; 800/281

(57) ABSTRACT

This invention relates to cDNA sequences encoding Δ5-fatty acid desaturases comprising the sequences shown in SEQ.1 and SEQ.2.

This invention relates to DNA sequences encoding Δ5-fatty acid desaturases, the encoded Δ5-fatty acid desaturases, and applications for the Δ5-fatty acid desaturases.

Polyunsaturated fatty acids are important neutraceutically due to their specific health promoting activities, and biomedically in respect of their potential pharmaceutical applications in the treatment of specific disease conditions.

Polyunsaturated fatty acids are the precursors for two major classes of metabolites: prostanoids (which include prostaglandins and thromboxanes) and leukotrienes. Δ5-fatty acid desaturase catalyses the conversion of dihomogammna linolenic acid (DHL) to arachidonic (AA) acid, and eicosatetraenoate (ETA) to ecosapentaenoate (EPA), by the introduction of double bonds at the Δ5 carbon of the respective substrates, and exists as an endoplasmic reticulum membrane-bound protein in its native state.

Arachidonic acid has a 20 carbon chain with 4 double bonds and is of great importance in human metabolism since it is a precursor for the synthesis of prostaglandins—20-carbon chain fatty acids that contain a 5 carbon ring. Prostaglandins are modulators of hormone action and the potential effects of prostaglandins include the stimulation of inflammation, the regulation of blood flow to particular organs, the control of ion transport across some membranes, and the modulation of synaptic transmission. Prostaglandins are also potentially useful as contraceptives due to their ability to suppress progesterone secretion. Therefore, the ability to modulate prostaglandin synthesis by controlled levels of expression of polyunsaturated fatty acid precursor synthesis is very important both medically and commercially.

The increased importance of polyunsaturated fatty acids in the food and pharmaceutical industries has led to an increased demand which has exceeded current production levels and supplementary sources of high quality, low cost polyunsaturated fatty acids are required.

Current commercial sources of polyunsaturated fatty acids include selected seed plants, marine fish and selected mammals, and traditional processing techniques for extracting the polyunsaturated fatty acids from these sources include solvent extraction, winterization, urea-adduct formation and distillation. However, present sources have the disadvantages of seasonal and climatic variations in both production levels and quality, a lack of availability of plant and fish sources, and the high costs of refining low-grade oils. High costs coupled with insufficient production levels have retarded the development of commercially exploitable applications of polyunsaturated fatty acids.

Much effort has gone into developing alternative sources of polyunsaturated fatty acids, and studies have been carried out to characterise the constituent genes and encoded proteins of their biosynthesis. The engineering of polyunsaturated fatty acid biosynthesis into oilseeds for example has many advantages for the production of large scale quantities of, for example, γ-linolenate (GLA), dihomo-γ(-linolenate (DHGLA), arachidonic acid (AA), eicosapentaenoate (EPA) and docosahezaenoate (DHA). The practicality of this has been illustrated by the expression of a Borage Δ6 desaturase gene in tobacco resulting in the production of GLA and the octadecatetraenoic acid, 18:4 (Soyanova et al (1997), PNAS 94, 9411-9414). As more of the biosynthetic genes for polyunsaturated fatty acid synthesis become available, this opens up the possibility of producing at least GLA, AA, EPA and DHA in oil seeds, as well as controlling the type of lipid assembled. Benefits which would be obtained from such crops include a cheap and sustainable supply of desirable polyunsaturated fatty acids on a large scale, tailored polyunsaturated fatty acids profiles to meet specific nutritional requirements, and in the fine chemical industry, the production of unusual fatty acids with prescribed levels and locations of unsaturation.

A further approach to the production of polyunsaturated fatty acids is to utilise the biosynthetic capacity of lower organisms e.g. algae, bacteria, fungi (including phycomycetes) which can synthesise the entire range of polyunsaturated fatty acids and can be grown on an industrial scale. Genetic transformation of these organisms will enable the development of overproducing strains and the manipulation of the polyunsaturated profile by pathway engineering.

Fungal Δ5 and Δ6 fatty acid desaturases have been cloned, and their sequences disclosed in WO98/46763, WO98/46764 and WO98/46765.

Polyunsaturated fatty acid metabolism is of greatest importance in human metabolism. These acids, via the eicosanoids, are fundamental to the proper maintenance of homeostasis and are linked to serious physiological and pathophysiological syndromes.

The inventors have surprisingly isolated and characterised a DNA sequence from the soil-borne filamentous fungus of the zygomycete class Mortierella alpina encoding a functional Δ5-fatty acid desaturase.

In addition, the inventors have surprisingly isolated and characterised a DNA sequence from the nematode worm, Caenorhabditis elegans encoding a functional Δ5-fatty acid desaturase. This DNA sequence, encoding a functional Δ5-fatty acid desaturase is thought likely to be more closely related to the human Δ5-fatty acid desaturase than any of the Δ5-fatty acid desaturase gene sequences isolated so far.

As well as the potential human benefits from the polypeptide encoded by the DNA sequences of this invention, the DNA sequences of this invention may enable the cloning of the equivalent human gene and thereby facilitate overproduction of the human DNA sequence and allow its biomedical exploitation in the treatment of certain human diseases.

Plant and fungal desaturases are mainly integral membrane polypeptides which makes them difficult to purify and subsequently characterise by conventional methods. Hence, molecular techniques including the use of mutants and transgenic plants have been adopted in order to better study lipid metabolism.

A first aspect of the invention provides an isolated animal Δ5-fatty acid desaturase and functional portions thereof.

A second aspect of the invention provides an isolated C. elegans Δ5-fatty acid desaturase.

A third aspect of the invention provides a DNA sequence according to a first or second aspect of the invention comprises at least a portion of the sequence shown in SEQ.2 and equivalents to that sequence, or to portions of that sequence, which encode a functional Δ5-fatty acid desaturase by virtue of the degeneracy of the genetic code. Preferably, the DNA sequence is derived from a Caenorhabditis elegans DNA sequence.

Preferably, the gene encoding the Δ5-fatty acid desaturase encoded by the cloned gene is 1341 bp long. The protein is 447 amino acids long with an estimated molecular weight of 57 kDa.

Alternatively, the DNA sequence encodes a functional Δ5-fatty acid desaturase and comprises at least a portion of the sequence shown in SEQ.1 and equivalents to that sequence, or to portions of that sequence, which encode a functional Δ5-fatty acid desaturase by virtue of the degeneracy of the genetic code. Preferably, the DNA sequence is derived from a Mortierella alpina DNA sequence.

Preferably, the gene encoding the Δ5-fatty acid desaturase encoded by the cloned gene is 1338 bp long. The protein is 446 amino acids long with an estimated molecular weight of 57 kDa.

Preferably, a DNA sequence according to a third aspect of the invention is functional in a mammal.

Preferably, the DNA sequence is expressed in a mammal.

Preferably, the DNA sequence is expressed in a human.

Preferably, the DNA sequence is obtained by modification of a functional natural gene encoding a Δ-5 fatty acid desaturase.

Preferably, the modification includes modification by chemical, physical, or biological means without removing a catalytic activity of the enzyme which it encodes.

Preferably, the modification improves a catalytic activity of the enzyme which it encodes.

Preferably, the biological modification includes recombinant DNA methods and forced evolution techniques.

Preferably, the forced evolution technique is DNA shuffling.

A fourth aspect of the invention provides a polypeptide encoded by a DNA sequence according to a third aspect of the invention.

Preferably, at least a portion of the polypeptide has the sequence shown in SEQ.3 or functional equivalents to that sequence or portions of that sequence. Alternatively, at least a portion of the polypeptide has the sequence shown in SEQ.4 or functional equivalents to that sequence or portions of that sequence.

Preferably, the polypeptide catalyses the conversion of dihomogamma linolenic acid to arachidonic acid.

Preferably, the polypeptide has been modified without removing the catalytic activity of the encoded polypeptide.

Preferably, the polypeptide has been modified in such a way as to introduce a specific level of saturation of a substrate at a specific location within the molecular structure of the substrate.

A fifth aspect of the invention provides a vector containing a DNA sequence of any portion of a DNA sequence according to a third aspect of the invention..

A sixth aspect of the invention provides a method of producing polyunsaturated fatty acids comprising contacting a substrate with a Δ5-fatty acid desaturase according to a first or second aspect of the invention, or a polypeptide according to a fourth aspect of the invention.

A seventh aspect of the invention provides a method of converting dihomogamma linoleic acid to arachidonic acid wherein the conversion is catalysed by a Δ5-fatty acid desaturase according to a first or second aspect of the invention, or a polypeptide according to a fourth aspect of the invention.

An eighth aspect of the invention provides an organism engineered to produce high levels of a polypeptide according to a fourth aspect of the invention.

A ninth aspect of the invention provides an organism engineered to produce high levels of a product of a reaction catalysed by a Δ5-fatty acid desaturase according to a first or second aspect of the invention, or by a polypeptide according to a fourth aspect of the invention.

Preferably, the organism has been engineered to carry out the method according to a sixth or seventh aspect of the invention.

Preferably, the organism is a microorganism.

Preferably, the microorganism is selected from algae, bacteria and fungi.

Preferably, the fungi includes phycomycetes. Alternatively, the microorganism is a yeast.

Alternatively, the organism is a plant. Preferably, the plant is selected from oil seed plants.

Preferably, the oil seed plants are selected from oil seed rape, sunflower, cereals including maize, tobacco, legumes including peanut and soybean, safflower, oil palm, coconut and other palms, cotton, sesame, mustard, linseed, castor, borage and evening primrose.

A tenth aspect of the invention provides a seed or other reproductive material derived from an organism according to a ninth aspect of the invention.

Preferably, the organism is a mammal.

An eleventh aspect of the invention provides an isolated multienzyme pathway wherein the pathway includes a Δ5-fatty acid desaturase according to a first or second aspect of the invention.

A twelfth aspect of the invention provides a compound produced by a conversion of a substrate, wherein said conversion is catalysed by a Δ5-fatty acid desaturase according to a first or second aspect of the invention.

A thirteenth aspect of the invention provides an intermediate compound produced by the reaction catalysed by a Δ5-fatty acid desaturase according to a first or second aspect of the invention.

A fourteenth aspect of the invention provides a foodstuff or dietary supplement containing a polyunsaturated fatty acid produced by a method according to a sixth aspect of the invention.

A fifteenth aspect of the invention provides a pharmaceutical preparation containing a polyunsaturated fatty acid produced by a method according to a sixth aspect of the invention.

A sixteenth aspect of the invention provides prostaglandins synthesised by a biosynthetic pathway including a catalytic activity of a Δ5-fatty acid desaturase according to a first or second aspect of the invention.

A seventeenth aspect of the invention provides a method for the modulation of prostaglandins synthesis by the control of the levels of expression of a DNA sequence according to a third aspect of the invention.

An eighteenth aspect of the invention provides a probe comprising all or part of a DNA sequence according to a third aspect of the invention,or an equivalent RNA sequence.

A nineteenth aspect of the invention provides a probe comprising all or part of a Δ5-fatty acid desaturase polypeptide according to a fourth aspect of the invention.

A twentieth aspect of the invention provides a method of isolating Δ5-fatty acid desaturases using a probe according to a nineteenth aspect of the invention.

It is possible that the gene of the invention may be transformed into human cells and exploited in gene therapy techniques at a suitable level in vivo to provide a constant supply of enzyme converting fatty acids to polyunsaturated fatty acids within the patient's body. This could be an effective preventative treatment for example, in patients suffering high levels of cholesterol or other medical conditions where administration of polyunsaturated fatty acids may have beneficial disease-preventative effects.

In addition, either whole or part of the DNA sequences of the invention, or whole or part of the polypeptide sequences of the invention could be used as search probes for research or diagnostic purposes.

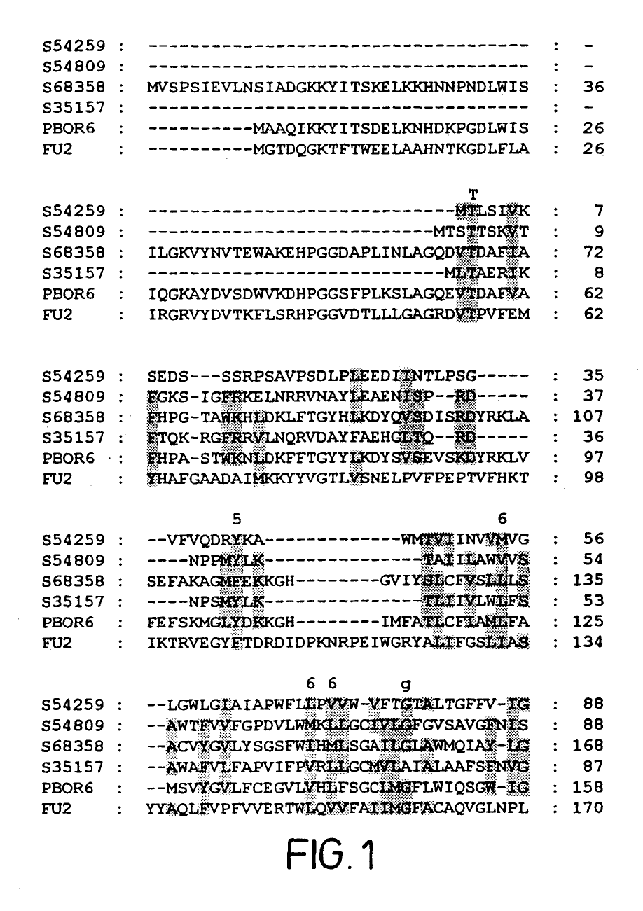

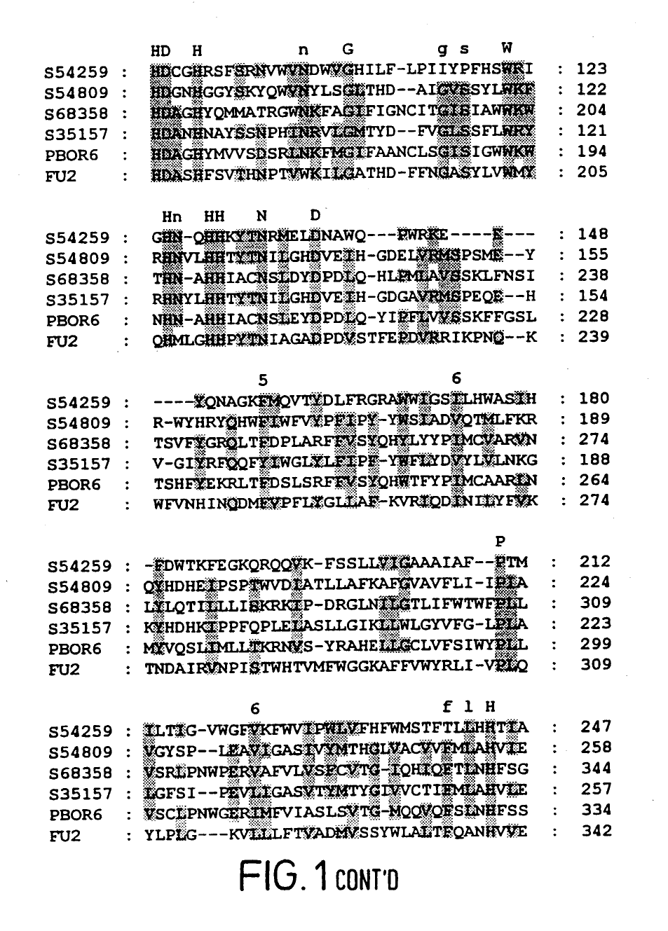

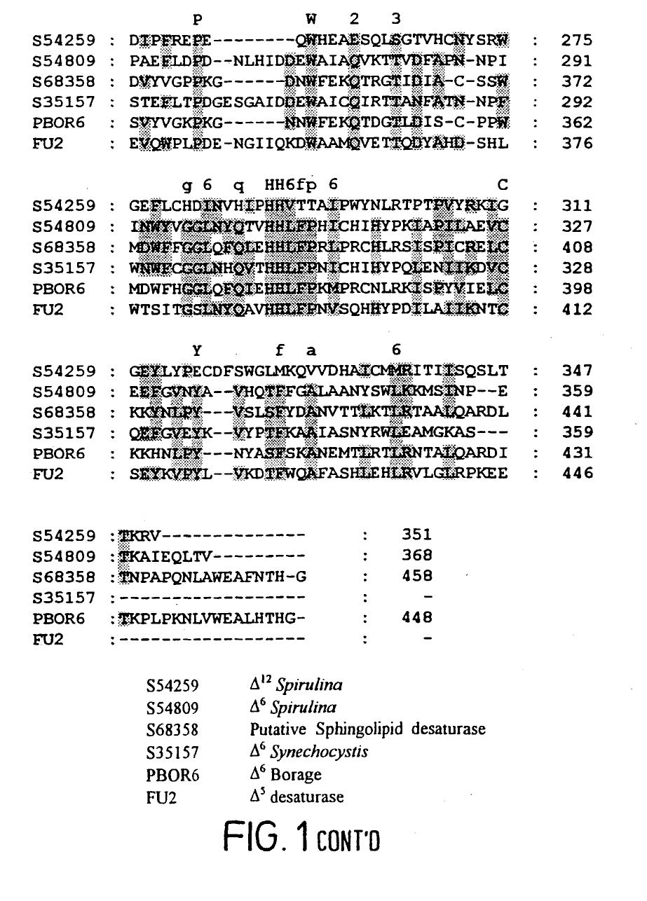

The invention will now be described by way of example only, with reference to the accompanying drawings, SEQ.1 to SEQ.4, and FIGS. 1 to 4, in which:

SEQ.1 is a cDNA sequence encoding Δ5-fatty acid desaturase from Mortierella alpina and;

SEQ.2 is a cDNA sequence encoding Δ5-fatty acid desaturase from C. elegans ; and

SEQ.3 is the peptide sequence obtained by translating the gene sequence of SEQ.1; and

SEQ.4 is the peptide sequence obtained by translating the gene sequence of SEQ2; and

FIG. 1 is a line-up of the gene encoding Mortierella alpina Δ5-fatty acid desaturase with various Δ6 desaturases and a Δ12 desaturase; and

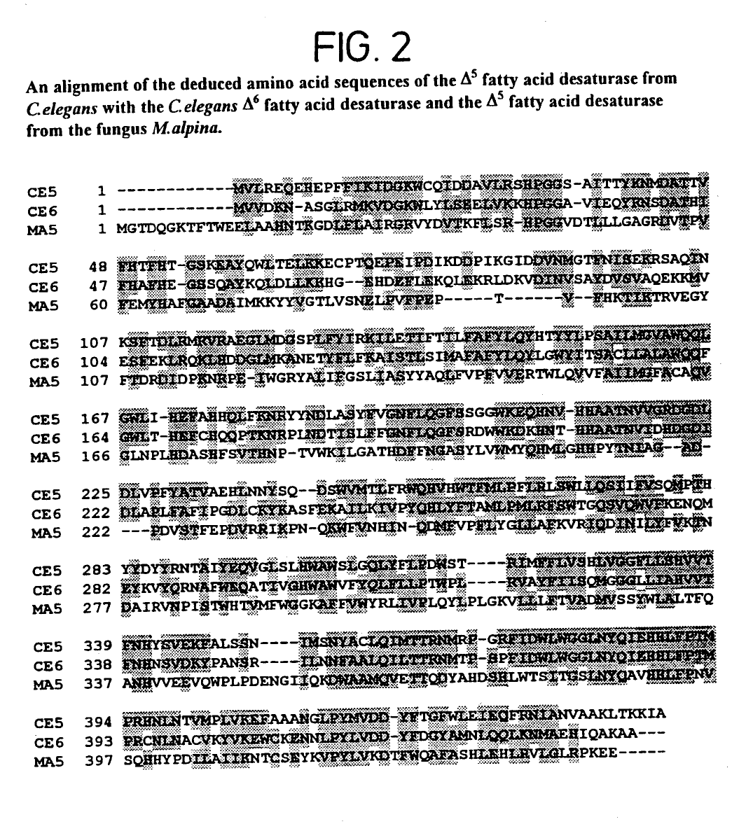

FIG. 2 is a line-up of the gene encoding Δ5-fatty acid desaturase with the C. elegans Δ6 desaturase and the fungal Δ5 desaturase from M. alpina; and

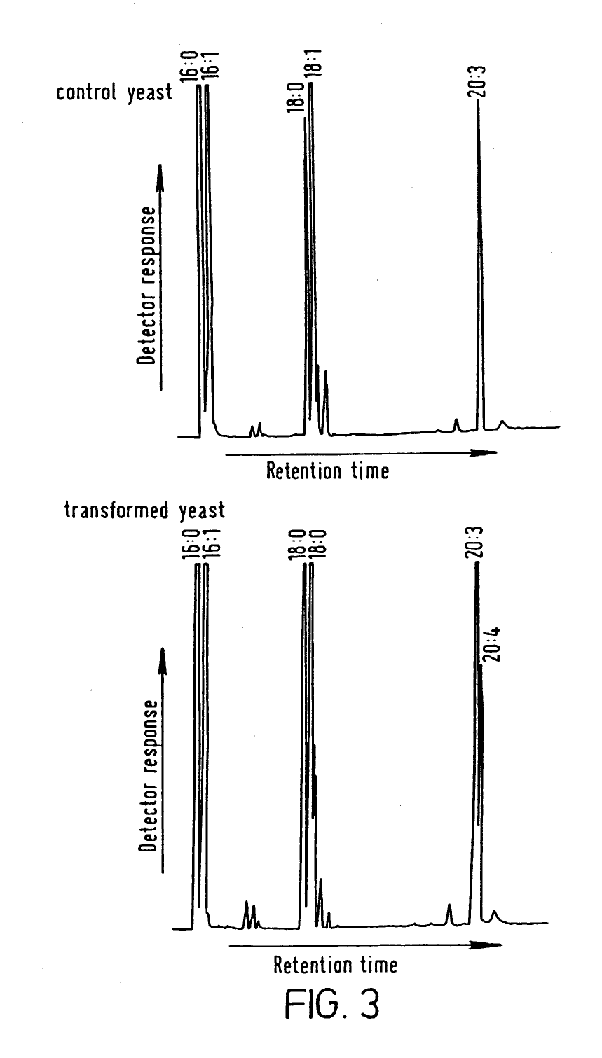

FIG. 3 is a gas chromatography trace of the fatty acid methyl esters of induced yeast cell transformants transformed with the Mortierella alpina Δ5-fatty acid desaturase gene and uninduced yeast cell transformants; and

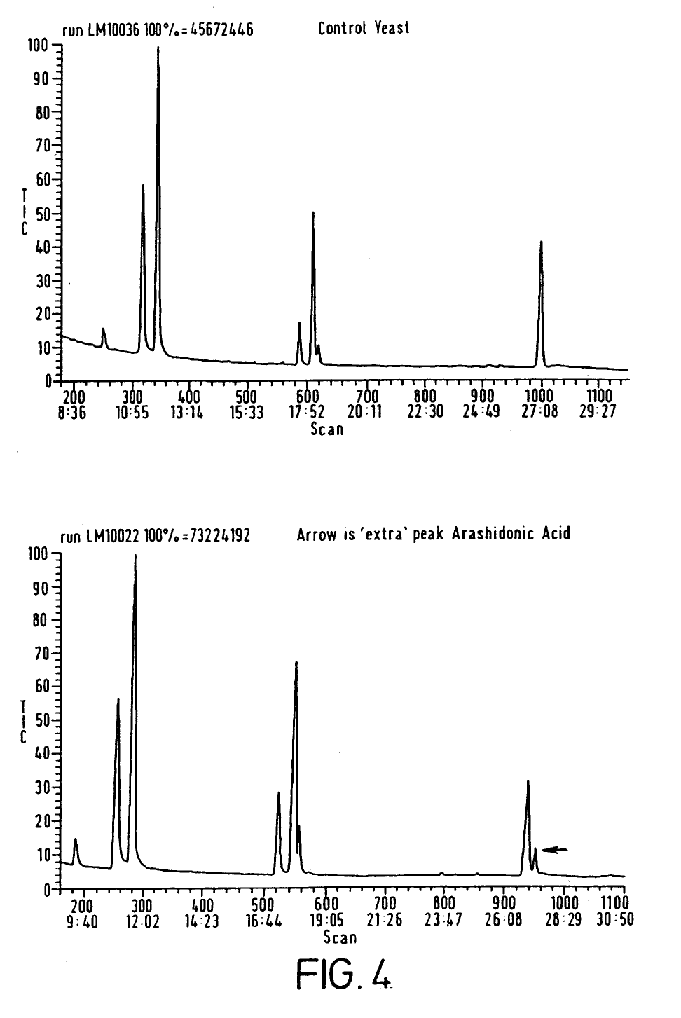

FIG. 4 is a gas chromatography trace of the fatty acid methyl esters of induced yeast cell transformants transformed with the C. elegans Δ5-fatty acid desaturase gene and uninduced yeast cell transformants.

Cloning and Sequencing of the Δ5-Fatty Acid Desaturase Gene from Mortierella alpina

The DNA sequences of the invention encode Δ5-fatty acid desaturases and were cloned using PCR technology in combination with cDNA library templates and specifically designed primers. The function of the DNA sequences, namely the conversion of dihanogamma linolenic acid (DHL) to arachidonic acid (AA), and eicostatetraenoate (ETA) to ecosapentaenoate (EPA), were verified by expressing the corresponding cDNAs in yeast.

The Δ5-fatty acid desaturase gene from Mortierella alpina was cloned by Polymerase Chain Reaction (PCR) techniques using cDNA from Mortierella alpina as the template and specifically designed degenerate oligonucleotide primers (DP) as shown below, based on the first and third histidine bases of plant Δ12 and Δ15 desaturases previously identified by Shanklin (Shanklin, J, Whittle, E J & Fox, B G. Biochemistry. 33, 12787-12794 (1994)). ps Degenerate Oligonucleotide Primers (DP)

5′-GCGAATTA(A/T)TIGGICA(T/C)GA(T/C)TG(T/C)GICA-3′
5′-GCGAATTCATIT(G/T)IGG(A/G)AAIA(G/A)(A/G)TG(A/G)T

G-3′

where I represents inosine, and the Eco RI sites are underlined.

The PCR amplifications were run entirely conventionally on a thermal cycler made by using a program of 2 minutes at 94° C. then 45 seconds at 94° C., 1 minute at 55° C. and 1 minute at 72° C. for 32 cycles followed by extension at 72° C. for a further 10 minutes. PCR amplification products were separated on 1% agarose gels.

The range of PCR products amplified from the Mortierella alpina cDNA template included a 660 bp product which was gel purified, cloned into pGEM-T (Promega®) and transformed into the Escherichia coli expression host, DH5α.

Primers (P) were designed against the 660 bp product sequence and fragment amplification carried out by PCR using the cloned 660 bp fragment as a template, and sequence-specific primers (P) based on the 660 bp product sequence.

| | |
|---|---|
| Delta B for | 5′-GATGCGTCTCACTTTTCA-3′ |
| Delta B rev. | 5′-GTGGTGCACAGCCTGGTAGTT-3′ |

The products of this PCR amplification were gel purified and used as probes to screen a Mortierella alpina cDNA library. The fragment probe hybridised to 25 out of the 3.5×10⁵ phage clones screened and one clone was shown, by restriction analysis, to have the expected size of 1.5 kb. This clone, designated L11, was selected for further analysis.

Sequence analysis of L11 revealed an open reading frame of 1,338 bp in length encoding a polypeptide of 446 amino acids. When analyzed on the protein and genomic databases using the GCG 8 Program (Devereux J. et al. Nucleic Acids. Res.. 12, 387-395 (1984)), L11 showed a low level 20% identity to the Δ6 desaturase gene from Synechocystis sp. PCC6803 (FIG. 1).

In FIG. 1, the sequences in the line-up have the following Accession numbers:

| | | |
|---|---|---|
| S54259 | Δ12 Spirulina | Accession number: X86736 |
| S54809 | Δ6 Spirulina | Accession number: X87094 |
| S68358 | Putative Sphingolipid desaturase | Accession number: X87143 |
| S35157 | Δ6 Synechocystis | Accession number: L11421 |
| PBOR6 | Δ6 Borage | Accession number U79010 |
| FU2 | Δ5 desaturase | Accession number AF054824 |

In addition, although all three histidine boxes characteristic of desaturase enzymes are present in the translated sequence, the third histidine box located at position 1159 bp in the sequence contains the variant QXXHH. The translated sequence also contains a cytochromc b₅-like heme-binding domain at the N-terminus which includes the EHPGG motif whereas previously, this feature has only been observed at the C-terminus of other fungal desaturases.

Southern Blotting of Genomic DNA

Sequence specific primers (P) designed against the L11 sequence between histidine boxes 1 and 3 of L11, were used in a PCR reaction to amplify a 660 bp region of the L11 sequence.

The 660 bp PCR product was gel purified and Southern blots of restricted Mortierella alpina and Mucor circinelloides genomic DNA carried out using the 660 bp fragment as a probe. The results suggest that the gene encoding the Δ5-fatty acid desaturase of the invention is present in single copy in Mortierella alpina and appears to be absent from Mucor circinelloides. In addition, there is no detectable Δ5-fatty acid desaturase activity in Mucor circinelloides.

Expression of the Cloned Mortierella alpina Gene Encoding Δ5-Fatty Acid Desaturase

In order to confirm that the L11 sequence encoded a Δ5-fatty acid desaturase enzyme, the cDNA was subcloned into the yeast expression vector, pYES2, supplied by Invitrogen™ under the control of the GAL4 polymerase promoter to yield plasmid pYES2/L11. The expression of L11 was checked by in vitro transcription-translation of pYES2/L11 using the Promega™ coupled Transcription and Translation system. ³⁵S methionine-labelled translation products were generated which were run on SDS PAGE and visualised by exposure to autoradiograph film. The estimated molecular weight of the product was 55-60 kD and a control plasmid, pYES2 with no insert, failed to yield any labelled translation product.

Construct, pYES2/L11, was transformed into yeast Saccharomyces cerevisiae and grown on uracil-deficient YCA medium. Transformants were selected by virtue of the presence of the URA3 selectable marker carried by pYES2/L11 and expression of L11 was induced by the addition of galactose to a final concentration of 1% mM. The cultures were grown overnight in the presence of 0.5 mM dihomo gamma linolenate, detergent (1% tergitol NP-40) and 2% raffinose. Aliquots were harvested at t=0, t=4 hours, and t=16 hours.

Yeast total fatty acids were analysed by GC of methyl esters. The lipids from the induced and uninduced control samples were transmethylated with 1M HCL in methanol at 80° C. for 1 hr. Fatty acid methyl esters (FAMES) were extracted in hexane. GC analysis of FAMES was conducted using a Hewlett Packard 58804 Series Gas Chromatograph equipped with a 25M×0.32 mm RSL-500 BP bonded capillary column and a flame ionization detector.

When methyl esters of the total fatty acids isolated from yeast carrying the plasmid pYes2/L11 and grown in the presence of galactose and dihomo gamma linolenic acid were analysed by GC an additional peak was observed (see FIG. 3). This extra peak had the same retention time as the authentic arachidonic acid standard (Sigma) indicating that the transgenic yeast were capable of desaturating Dihomo gamma linolenic acid at the Δ5 position. No such peaks were observed in any of the control samples (transformation with pYes2) FIG. 3. The identity of the additional peak was confirmed by GCMS (Kratos MS80RFA operating at an ionization voltage of 70 eV with a scan range of 500-40 daltons) which positively identified this compound as arachidonic acid.

This demonstrates that the DNA sequence from Mortierella alpina encodes a functional polypeptide involved in the synthesis of arachidonic acid in the presence of galactose and dihomo gamma linolenate.

Cloning and Sequencing of the C. elegans Δ5-Fatty Acid Desaturase Gene

Previously, the inventors identified fungal Δ5 and Δ6-fatty acid desaturases from both plant and animal species which were distinct from previously identified microsomal desaturases. This difference was due to the presence of an N-terminal extension which showed homology to the electron donor protein cytochrome b₅.

During the characterisation of the fungal (Mortierella alpina) Δ5-fatty acid desaturase and the C. elegans Δ6-fatty acid desaturase (present on cosmid W08D2 (Accession No. Z70271)), the inventors identified a related sequence on cosmid T13F2.1 (Accession No. Z81122) also containing C. elegans DNA likely to encode a fatty acid desaturase.

Analysis of the sequences (using Genefinder program (Wilson, R. et al (1994) Nature, 368, 32-38)) revealed that cosmids W08D2 and T13F2 contained overlapping regions. In addition, it was found that cosmid T13F2 contained an open reading frame (ORF), designated T13F2.1, which contained an N-terminal cytochrome b₅ domain (defined by the diagnostic His-Pro-Gly-Gly motif), as well as three ‘histidine boxes’ characteristic of all microsomal desaturases. Further, this putative desaturase contained a variant third histidine box, with a H→Q substitution for the first histidine in the His-X-X-His-His motif. This glutamate substitution is present in both plant and animal Δ6-fatty acid desaturases and in the fungal Δ5-fatty acid desaturase from M. alpina.

The overlap between cosmids T13F2 and W08D2 allowed the determination of the proximity of the putative desaturase ORF, T13F2.1, to the Δ6-fatty acid desaturase, revealing that the two sequences were arranged in tandem on chromosome IV, separated by 990 bases from the predicted stop codon of T13F2.1 to the initiating methionine triplet of the Δ6-fatty acid desaturase.

Since sequence analysis predicted that the T13F2.1 ORF was interspersed with a number of introns, heterologous functional expression of genomic DNA was unfeasible. Therefore, the polymerase chain reaction (PCR) was used to amplify a partial cDNA clone corresponding to a large predicted exon at the 5′ end of the T13F2.1 ORF using the following primers, CEFOR AND CEREV:

| | |
|---|---|
| CEFOR: | 5′- ATGGTATTACGAGAGCAAGA-3′ |
| CEREV: | 5′-TCTGGGATCTCTGGTTCTTG-3′ |

After initial denaturation at 94° C. for 2 minutes, amplification was performed in 32 cycles of: 45 seconds at 94° C., 1 minute at 55° C., and 1 minute at 72° C. followed by a final extension at 72° C. for a further 10 minutes.

A DNA fragment of the correct predicted size was amplified (as visualised on a 1% agarose gel), the gel band was cut out, the DNA purified and ligated directly into pGEM-T (Promega), and the resulting plasmid transformed into E. coli DH5α cells. Plasmid DNA was purified for sequencing using the Qiagen QIAprep miniprep kit, and the nucleotide sequence of the insert determined by automated sequencing using an ABI-377 DNA sequencer.

In order to isolate the complete coding region corresponding to ORF T13F2.1, this isolated 233 bp PCR-amplified fragment was used to screen a mixed stage C. elegans cDNA library that had been constructed in λZapII by Prof Yuji Kohara—Mishima, Japan. The screening was carried out using standard techniques (Sambrook et al (1989) Molecular Cloning. A Laboratory Manual) using the cloned PCR product as a probe. The DNA fragments were labelled with α [³²P] d CTP using the Ready to Go DNA-Labelling reaction mix (Pharmacia). Of 1.4×10⁵ pfu screened for hybridization to the 233 bp fragment, 5 plaques gave positive signals and were cored out of the agar plates and eluted into SM buffer. The resultant phage suspensions were screened for the presence of T13F2.1 by PCR amplification using CEFOR and CEREV. One clone, designated L4, was purified by 2 additional rounds of plating and hybridisation screening at 65° C. using the 233 bp fragment isolated by PCR. Plasmid L4 was released from λ clone L4 by excision and the cDNA insert sequenced on both strands using a Perkin Elmer AB1-377 DNA sequencer.

The resulting DNA sequence is shown in SEQ.2, and the predicted amino acid sequence is shown in SEQ.4.

Functional Analysis of L4 in Yeast

The complete coding region (coding for 447 amino acids) of L4 was amplified by PCR using the primers YCEDFor and TCEDRev shown below, which also introduced flanking HindIII and BamHI restriction sites:

YCEDFor:

5′-GCGAAGCTTAAAATGGTATTACGAGAGCAAGAGC-3′

(annealing to the initiating methionine is indicated by the bold type face and the Hind III restriction site is underlined)

YCEDRev:

5′-GCGGATCCAATCTAGGCAATCTTTTAGTCAA-3′

The amplified PCR product containing the complete coding region of L4 was ligated into the yeast expression vector, pYES2 (Invitrogen), downstream of the GAL1 promoter using HindIII and BamIII restriction sites (enzymes supplied by Boehringer Mannheim). The resulting construct, designated pYES2/L4, was transformed into E. coli, and the fidelity of the PCR-generated insert in plasmid pYES2/L4 was confirmed in vitro by coupled transcription/translation using the TNT system (Promega). The resulting translation products were labelled with ³⁵S methionine, separated by SDS-PAGE and visualised by autoradiography.

The translation product obtained from pYES2/L4 had a molecular weight of approximately Mᵣ57,000, whereas the control vector, pYES2 with no insert, did not yield a translation product.

For functional analysis of the L4 coding region the recombinant plasmid was transformed into S. cerevisiae DBY746 by the lithium acetate method (Elble R. (1992) Bio Techniques 13 18-20). Cells were cultured overnight in a medium containing raffinose as a carbon source, and supplemented by the addition of either linoleic acid (18:2 Δ^{9,12}) or di-homo-γ-linolenic acid (C20:3 Δ^{8,11,14}) in the presence of 1% tergitol (as described by Napier et al (1998) Biochem. J. 330 611-614). These fatty acids are not present in S. cerevisiae but serve as the specific substrates for either the Δ⁶ or Δ⁵-desaturase, respectively. Expression of the L4 coding region from the GAL1 promoter of the vector was induced by the addition of galactose to 1%. Growth of the cultures was continued for 16 hours before removal of aliquots for the analysis of fatty acids by GC Total fatty acids extracted from yeast cultures were analysed by gas chromatography (GC) of methyl esters. Lipids were transmethylated with 1M HCl in methanol at 80° C. for 1 hr, then fatty acid methyl esters (FAMEs) were extracted in hexane. GC analysis of FAMEs were conducted using a Hewlett Packard 5880A Series Gas chromatograph equipped with a 25 M×0.32 mm RSL-500 BP bonded capillary column and a flame ionization detector. Fatty acids were identified by comparison with retention times of FAME standards (Sigma). Relative percentages of the fatty acids were estimated from peak areas. Arachidonic acid was identified by GC-MS using a Krats MS80RFA operating at an ionization voltage of 70 eV, with a scan range of 500-40 daltons. FIG. 4 shows the result of GC analysis of the fatty acid methyl esters of transformed yeast strains. An additional peak is apparent in the trace obtained from induced pYES2/L4 grown in the presence of di-homo-γ-linolenic acid compared to an empty-vector control. This peak was also absent from uninduced cultures grown on di-homo-γ-linolenic acid and it is also important to note that pYES2/L4 grown in the presence of linoleic acid failed to accumulate any novel peaks indicating that this fatty acid is not a substrate for the enzyme encoded by the C. elgans cDNA. The retention time of the additional peak is identical to that of the authentic methyl-arachidonic acid standard. The fatty acid produced from di-homo-γ-linolenic acid was further characterised by GCMS (Gas Chromatography Mass Spectrometry) and identified as arachidonic acid. The results show, therefore, that yeast cells transformed with the plasmid pYES2/L4 had acquired functional Δ⁵-desaturase activity and were now capable of synthesising arachidonic acid from the substrate di-homo-γ-linolenic acid. The Δ⁵-desaturase in the transformed yeast appeared to be an efficient catalyst.

This demonstrates that the DNA sequence from C. elegans encodes a functional polypeptide involved in the synthesis of arachidonic acid in the presence of galactose and di-homo-γ-linolenate.

| | | |
|---|---|---|
| 1 | ATGGTATTAC GAGAGCAAGA GCATGAGCCA TTCTTCATTA AAATTGATGG | SEQ. 2 |
| 51 | AAAATGGTGT CAAATTGACG ATGCTGTCCT GAGATCACAT CCAGGTGGTA | |
| 101 | GTGCAATTAC TACCTATAAA AATATGGATG CCACTACCGT ATTCCACACA | |
| 151 | TTCCATACTG GTTCTAAAGA AGCGTATCAA TGGCTGACAG AATTGAAAAA | |
| 201 | AGAGTGCCCT ACACAAGAAC CAGAGATCCC AGATATTAAG GATGACCCAA | |
| 251 | TCAAAGGAAT TGATGATGTG AACATGGGAA CTTTCAATAT TTCTGAGAAA | |
| 301 | CGATCTGCCC AAATAAATAA AAGTTTCACT GATCTACGTA TGCGAGTTCG | |
| 351 | TGCAGAAGGA CTTATGGATG GATCTCCTTT GTTCTACATT AGAAAAATTC | |
| 401 | TTGAAACAAT CTTCACAATT CTTTTTGCAT TCTACCTTCA ATACCACACA | |
| 451 | TATTATCTTC CATCAGCTAT TCTAATGGGA GTTGCGTGGC AACAATTGGG | |
| 501 | ATGGTTAATC CATGAATTCG CACATCATCA GTTGTTCAAA AACAGATACT | |
| 551 | ACAATGATTT GGCCAGCTAT TTCGTTGGAA ACTTTTTACA AGGATTCTCA | |
| 601 | TCTGGTGGTT GGAAAGAGCA GCACAATGTG CATCACGCAG CCACAAATGT | |
| 651 | TGTTGGACGA GACGGAGATC TTGATTTAGT CCCATTCTAT GCTACAGTGG | |
| 701 | CAGAACATCT CAACAATTAT TCTCAGGATT CATGGGTTAT GACTCTATTC | |
| 751 | AGATGGCAAC ATGTTCATTG GACATTCATG TTACCATTCC TCCGTCTCTC | |
| 801 | GTGGCTTCTT CAGTCAATCA TTTTTGTTAG TCAGATGCCA ACTCATTATT | |
| 851 | ATGACTATTA CAGAAATACT GCGATTTATG AACAGGTTGG TCTCTCTTTG | |
| 901 | CACTGGGCTT GGTCATTGGG TCAATTGTAT TTCCTACCCG ATTGGTCAAC | |
| 951 | TAGAATAATG TTCTTCCTTG TTTCTCATCT TGTTGGAGGT TTCCTGCTCT | |
| 1001 | CTCATGTAGT TACTTTCAAT CATTATTCAG TGGAGAAGTT TGCATTGAGC | |
| 1051 | TCGAACATCA TGTCAAATTA CGCTTGTCTT CAAATCATGA CCACAAGAAA | |
| 1101 | TATGAGACCT GGAAGATTCA TTGACTGGCT TTGGGGAGGT CTTAACTATC | |
| 1151 | AGATTGAGCA CCATCTTTTC CCAACGATGC CACGACACAA CTTGAACACT | |
| 1201 | GTTATGCCAC TTGTTAAGGA GTTTGCAGCA GCAAATGGTT TACCATACAT | |
| 1251 | GGTCGACGAT TATTTCACAG GATTCTGGCT TGAAATTGAG CAATTCCGAA | |
| 1301 | ATATTGCAAA TGTTGCTGCT AAATTGACTA AAAAGATTGC CTAG | |
| 1 | MGTDQGKTFT WEELAAHNTK GDLFLAIRGR VYDVTKFLSR HPGGVDTLLL | SEQ. 3 |
| 51 | GAGRDVTPVF EMYHAFGAAD AIMKKYYVGT LVSNELPVFP EPTVFHKTIK | |
| 101 | TRVEGYFTDR DIDPKNRPEI WGRYALIFGS LIASYYAQLF VPFVVERTWL | |
| 151 | QVVFAIIMGF ACAQVGLNPL HDASHFSVTH NPTVWKILGA THDFFNGASY | |
| 201 | LVWMYQHMLG HHPYTNIAGA DPDVSTFEPD VRRIKPNQKW FVNHINQDMF | |
| 251 | VPFLYGLLAF KVRIQDINIL YFVKTNDAIR VNPISTWHTV MFWGGKAFFV | |
| 301 | WYRLIVPLQY LPLGKVLLLF TVADMVSSYW LALTFQANHV VEEVQWPLPD | |
| 351 | ENGIIQKDWA AMQVETTQDY AHDSHLWTSI TGSLNYQAVH HLFPNVSQHH | |
| 401 | YPDILAIIKN TCSEYKVPYL VKDTFWQAFA SHLEHLRVLG LRPKEE* | |

The predicted amino acid sequence of L4 the gene
which encodes a Δ⁵ fatty acid desaturase
from C. elegans.

| | | |
|---|---|---|
| 1 | MVLREQEHEP FFIKIDGKWC QIDDAVLRSH PGGSAITTYK NMDATTVFHT | SEQ 4 |
| 51 | FHTGSKEAYQ WLTELKKECP TQEPEIPDIK DDPIKGIDDV NMGTFNISEK | |
| 101 | RSAQINKSFT DLRMRVRAEG LMDGSPLFYI RKILETIFTI LFAFYLQYHT | |
| 151 | YYLPSAILMG VAWQQLGWLI HEFAHHQLFK NRYYNDLASY FVGNFLQGFS | |
| 201 | SGGWKEQHNV HHAATNVVGR DGDLDLVPFY ATVAEHLNNY SQDSWVMTLF | |
| 251 | RWQHVHWTFM LPFLRLSWLL QSIIFVSQMP THYYDYYRNT AIYEQVGLSL | |
| 301 | HWAWSLGQLY FLPDWSTRIM FFLVSHLVGG FLLSHVVTFN HYSVEKFALS | |
| 351 | SNIMSNYACL QIMTTRNMRP GRFIDWLWGG LNYQIEHHLP PTMPRHNLNT | |
| 401 | VMPLVKEFAA ANGLPYMVDD YFTGFWLEIE QFRNIANVAA KLTKKIA* | |

1. An isolated animal Δ5-fatty acid desaturase and functional portions thereof.

2. Isolated C. elegans Δ5-fatty acid desaturase.

3. A DNA sequence encoding a Δ5-fatty acid desaturase according to claim 1 or claim 2.

4. A DNA sequence according to claim 3 and comprising at least a portion of the sequence shown in SEQ.2 and equivalents to that sequence, or to portions of that sequence, which encode a functional Δ5-fatty acid desaturase by virtue of the degeneracy of the genetic code.

5. A DNA sequence according to claim 4 derived from a Caenorhabditis elegans DNA sequence.

6. A DNA sequence according to claim 3 encoding a functional Δ5-fatty acid desaturase and comprising at least a portion of the sequence shown in SEQ.1 and equivalents to that sequence, or to portions of that sequence, which encode a functional Δ5-fatty acid desaturase by virtue of the degeneracy of the genetic code.

7. A DNA sequence according to claim 6 derived from a Mortierella alpina DNA sequence.

8. A DNA sequence according to any one of claims 3 to 7 wherein the DNA sequence is functional in a mammal.

9. A DNA sequence according to claim 8 in which the DNA sequence is expressed in a mammal

10. A DNA sequence according to claim 9 wherein the DNA sequence is expressed in a human.

11. A DNA sequence obtained by modification of a functional natural gene encoding a Δ-5 fatty acid desaturase according to claim 1 or claim 2.

12. A DNA sequence according to claim 11 wherein the modification includes modification by chemical, physical, or biological means without removing a catalytic activity of the enzyme which it encodes.

13. A DNA sequence according to claim 12 wherein the modification improves a catalytic activity of the enzyme which it encodes.

14. A DNA sequence according to claim 12 or 13 wherein the biological modification includes recombinant DNA methods and forced evolution techniques.

15. A DNA sequence according to claim 14 wherein the forced evolution technique is DNA shuffling.

16. A polypeptide encoded by a DNA sequence according to any of claims 3 to 15.

17. A polypeptide according to claim 16 wherein at least a portion of the polypeptide has the sequence shown in SEQ.3 or functional equivalents to that sequence or to portions of that sequence.

18. A polypeptide according to claim 16 wherein at least a portion of the polypeptide has the sequence shown in SEQ.4 or functional equivalents to that sequence or to portions of that sequence.

19. A polypeptide according to any of claims 16 to 18 wherein the polypeptide catalyses the conversion of dihomogamma linolenic acid to arachidonic acid.

20. A polypeptide according to any of claims 16 to 19 wherein the polypeptide has been modified without removing the catalytic activity of the encoded polypeptide.

21. A polypeptide according to claim 20 wherein the polypeptide has been modified in such a way as to introduce a specific level of saturation of a substrate at a specific location within the molecular structure of the substrate.

22. A vector containing a DNA sequence or any portion of a DNA sequence according to any of claims 3 to 15.

23. A method of producing polyunsaturated fatty acids comprising contacting a suitable substrate with a Δ5-fatty acid desaturase according to claim 1 or 2 or a polypeptide according to claim 16 to 21.

24. A method of converting dihomogamma linolenic acid to arachidonic acid wherein said conversion is catalysed by a Δ5-fatty acid desaturase according to claim 1 or 2 or a polypeptide or modified polypeptide according to any of claims 16 to 21.

25. An organism engineered to produce high levels of a polypeptide according to any of claims 16 to 21.

26. An organism engineered to produce high levels of a product of a reaction catalysed by a Δ5-fatty acid desaturase according to claim 1 or 2 or by a polypeptide according to any one of claims 16 to 21.

27. An organism which has been engineered to carry out the method of claim 23 or claim 24.

28. An organism according to either of claims 26 and 27 wherein the organism is a microorganism.

29. An organism according to claim 28 wherein a microorganism is selected from algae, bacteria and fungi.

30. An organism according to claim 29 wherein a fungi includes phycomycetes.

31. An organism according to claim 28 wherein said microorganism is a yeast.

32. An organism according to any of claims 25 to 27 wherein the organism is a plant.

33. An organism according to claim 32 wherein the plant is selected from oil seed plants and tobacco.

34. An organism according to claim 33 wherein the oil seed plants are selected from oil seed rape, sunflower, cereals including maize, tobacco, legumes including peanut and soybean, safflower, oil palm, coconut and other palms, cotton, sesame, mustard, linseed, castor, borage and evening primrose.

35. A seed or other reproductive material derived from an organism according to claim 33 or claim 34.

36. An organism according to any of claims 25 to 27 wherein the organism is a mammal.

37. An isolated multienzyme pathway wherein the pathway includes a Δ5 desaturase according to claim 1 or 2 or a polypeptide according to any of claims 16 to 21

38. A compound produced by a conversion of a substrate, wherein said conversion is catalysed by a Δ5 desaturase according to claim 1 or 2 or by a polypeptide according to any of claims 16 to 21.

39. An intermediate compound produced by the reaction catalysed by a Δ5 desaturase according to claim 1 or 2 or by a polypeptide according to any of claims 16 to 21.

40. A foodstuff or dietary supplement containing a polyunsaturated fatty acid produced by a method according to claim 23 or 24.

41. A pharmaceutical preparation containing a polyunsaturated fatty acid produced by a method according to claim 23 or 24.

42. Prostaglandins synthesised by a biosynthetic pathway including a catalytic activity of a Δ5 desaturase according to claim 1 or 2 or by a polypeptide according to any of claims 16 to 21.

43. A method for modulation of prostaglandin synthesis by the control of the levels of expression of a DNA sequence according to any of claims 3 to 15.

44. A probe comprising all or part of a DNA sequence according to any of claims 3 to 15 or an equivalent RNA sequence.

45. A diagnostic or search probe comprising all or part of a Δ5 desaturase according to claim 1 or 2 or of a polypeptide according to any of claims 16 to 21.

46. A method of isolating Δ5 desaturases using a probe according to claim 44 or 45.


SEQUENCE LISTING (23 sequences)

SEQ ID NO: 1 - DNA, 1405 bp, Mortierella alpina
atgggtacggaccaaggaaaaaccttcacctgggaagagctagcggcccataacaccaagggcgacctttttttggccatccgcggcagggtgtacgatgtcacaaagttcttgagccgccatcctggtggagtggacactctcctgctcggagctggccgagatgttactccggtctttgagatgtatcacgcgtttggggctgcagatgccatcatgaagaagtactatgtcggtacattggtttcgaatgagctgcccgtcttcccggagccaacggtgttccacaaaaccatcaagacgagagttgagggctactttacggatcgggacattgatcccaagaacagaccagagatctggggacgatacgctcttatctttggatccttgatcgcttcctactacgcgcagctctttgtgcctttcgttgtcgaacgcacatggctccaggtggtgtttgcaatcatcatgggatttgcgtgcgcacaagtcggactcaaccctcttcatgatgcgtctcacttttcagtgacccacaaccccactgtctggaagattctgggagccacgcacgactttttcaacggagcatcgtacctggtgtggatgtaccaacatatgctcggccatcacccctacaccaacattgctggagcagatcctgacgtgtcgacgtttgagcccgatgttcgtcgtatcaagcccaaccaaaagtggtttgttaaccacatcaaccaggacatgtttgttcctttcctgtacggactgctggcgttcaaggtgcgcattcaggacatcaacattttgtactttgtcaagacaaatgacgcaattcgcgtcaatcccatctcgacatggcacactgtgatgttctggggcggcaaggctttctttgtctggtatcgcctgattgttcccctgcagtatctgcccctgggcaaggtgctgctcctgttcacggtcgcggacatggtgtcgtcttactggctggcgctgaccttccaggcgaaccacgttgttgaggaagttcagtggccgttgcctgacgagaacgggatcatccaaaaggactgggcagctatgcaggttgagactacgcaggattacgcacacgactcgcacctctggaccagcattactggcagcttgaactaccaggctgtgcaccatctgttccccaacgtgtcgcagcaccattatcccgatattctggccatcatcaagaacacctgcagcgagtacaaggttccataccttgtcaaggataccttttggcaagcatttgcttcacatttggagcacttgcgtgttcttggactccgtcccaaggaagagtagaaagaaaaaaagcgccgaacgaagtattgccccctttttctccaagaaaaaaaaaaaaaaaaaa

SEQ ID NO: 2 - DNA, 1344 bp, C. elegans
atggtattacgagagcaagagcatgagccattcttcattaaaattgatggaaaatggtgtcaaattgacgatgctgtcctgagatcacatccaggtggtagtgcaattactacctataaaaatatggatgccactaccgtattccacacattccatactggttctaaagaagcgtatcaatggctgacagaattgaaaaaagagtgccctacacaagaaccagagatcccagatattaaggatgacccaatcaaaggaattgatgatgtgaacatgggaactttcaatatttctgagaaacgatctgcccaaataaataaaagtttcactgatctacgtatgcgagttcgtgcagaaggacttatggatggatctcctttgttctacattagaaaaattcttgaaacaatcttcacaattctttttgcattctaccttcaataccacacatattatcttccatcagctattctaatgggagttgcgtggcaacaattgggatggttaatccatgaattcgcacatcatcagttgttcaaaaacagatactacaatgatttggccagctatttcgttggaaactttttacaaggattctcatctggtggttggaaagagcagcacaatgtgcatcacgcagccacaaatgttgttggacgagacggagatcttgatttagtcccattctatgctacagtggcagaacatctcaacaattattctcaggattcatgggttatgactctattcagatggcaacatgttcattggacattcatgttaccattcctccgtctctcgtggcttcttcagtcaatcatttttgttagtcagatgccaactcattattatgactattacagaaatactgcgatttatgaacaggttggtctctctttgcactgggcttggtcattgggtcaattgtatttcctacccgattggtcaactagaataatgttcttccttgtttctcatcttgttggaggtttcctgctctctcatgtagttactttcaatcattattcagtggagaagtttgcattgagctcgaacatcatgtcaaattacgcttgtcttcaaatcatgaccacaagaaatatgagacctggaagattcattgactggctttggggaggtcttaactatcagattgagcaccatcttttcccaacgatgccacgacacaacttgaacactgttatgccacttgttaaggagtttgcagcagcaaatggtttaccatacatggtcgacgattatttcacaggattctggcttgaaattgagcaattccgaaatattgcaaatgttgctgctaaattgactaaaaagattgcctag

SEQ ID NO: 3 - protein, 446 aa, Mortierella alpina
MGTDQGKTFTWEELAAHNTKGDLFLAIRGRVYDVTKFLSRHPGGVDTLLLGAGRDVTPVFEMYHAFGAADAIMKKYYVGTLVSNELPVFPEPTVFHKTIKTRVEGYFTDRDIDPKNRPEIWGRYALIFGSLIASYYAQLFVPFVVERTWLQVVFAIIMGFACAQVGLNPLHDASHFSVTHNPTVWKILGATHDFFNGASYLVWMYQHMLGHHPYTNIAGADPDVSTFEPDVRRIKPNQKWFVNHINQDMFVPFLYGLLAFKVRIQDINILYFVKTNDAIRVNPISTWHTVMFWGGKAFFVWYRLIVPLQYLPLGKVLLLFTVADMVSSYWLALTFQANHVVEEVQWPLPDENGIIQKDWAAMQVETTQDYAHDSHLWTSITGSLNYQAVHHLFPNVSQHHYPDILAIIKNTCSEYKVPYLVKDTFWQAFASHLEHLRVLGLRPKEE

SEQ ID NO: 4 - protein, 448 aa, C. elegans
MVLREQEHEPFFIKIDGKWCQIDDAVLRSHPGGSAITTYKNMDATTVFHTFHTGSKEAYQWLTEKLKKECPTQEPEIPDIKDDPIKGIDDVNMGTFNISEKRSAQINKSFTDLRMRVRAEGLMGDSPLFYIRKILETIFTILFAFYLQYHTYYLPSAILMGVAWQQLGWLIHEFAHHQLFKNRYYNDLASYFVGNFLQGFSSGGWKEQHNVHHAATNVVGRDGDLDLVPFYATVAEHLNNYSQDSWVMTLFRWQHVHWTFMLPFLRLSWLLQSIIFVSQMPTHYYDYYRNTAIYEQVGLSLHWAWSLGQLYFLPDWSTRIMFFLVSHLVGGFLLSHVVTFNHYSVEKFALSSNIMSNYACLQIMTTRNMRPGRFIDWLWGGLNYQIEHHLFPTMPRHNLNTVMPLYKEFAAANGLPYMVDDYFTGFWLEIEQFRNIANVAAKLTKKIA

SEQ ID NO: 5 - DNA, 24 bp, Artificial Sequence
  PCR primer
gcgaattawtggcaygaytgygca

SEQ ID NO: 6 - DNA, 25 bp, Artificial Sequence
  PCR primer
gcgaattcattkggraaarrtgrtg

SEQ ID NO: 7 - DNA, 18 bp, Artificial Sequence
  PCR primer
gatgcgtctcacttttca

SEQ ID NO: 8 - DNA, 21 bp, Artificial Sequence
  PCR primer
gtggtgcacagcctggtagtt

SEQ ID NO: 9 - protein, 351 aa, Spirulina
MTLSIVKSEDSSSRPSAVPSDLPLEEDIINTLPSGVFVQDRYKAWMTVIINVVMVGLGWLGIAIAPWFLLPVVWVFTGTALTGFFVIGHDCGHRSFSRNVWVNDWVGHILFLPIIYPFHSWRIGHNQHHKYTNRMELDNAWQPWRKEEYQNAGKFMQVTYDLFRGRAWWIGSILHWASIHFDWTKFEGKQRQQVKFSSLLVIGAAAIAFPTMILTIGVWGFVKFWVIPWLVFHFWMSTFTLLHHTIADIPFREPEQWHEAESQLSGTVHCNYSRWGEFLCHDINVHIPHHVTTAIPWYNLRTPTPVYRKIGGEYLYPECDFSWGLMKQVVDHAICMMRITIISQSLTTKRV

SEQ ID NO: 10 - protein, 368 aa, Spirulina
MTSTTSKVTFGKSIGFRKELNRRVNAYLEAENISPRDNPPMYLKTAIILAWVVSAWTFVVFGPDVLWMKLLGCIVLGFGVSAVGFNISHDGNHGGYSKYQWVNYLSGLTHDAIGVSSYLWKFRHNVLHHTYTNILGHDVEIHGDELVRMSPSMEYRWYHRYQHWFIWFVYPFIPYYWSIADVQTMLFKRQYHDHEIPSPTWVDIATLLAFKAFGVAVFLIIPIAVGYSPLEAVIGASIVYMTHGLVACVVFMLAHVIEPAEFLDPDNLHIDDEWAIAQVKTTVDFAPNNPIINWYVGGLNYQTVHHLFPHICHIHYPKIAPILAEVCEEFGVNYAVHQTFFGALAANYSWLKKMSINPETKAIEQLTV

SEQ ID NO: 11 - protein, 458 aa, Helianthus annus
MVSPSIEVLNSIADGKKYITSKELKKHNNPNDLWISILGKVYNVTEWAKEHPGGDAPLINLAGQDVTDAFIAFHPGTAWKHLDKLFTGYHLKDYQVSDISRDYRKLASEFAKAGMFEKKGHGVIYSLCFVSLLLSACVYGVLYSGSFWIHMLSGAILGLAWMQIAYLGHDAGHYQMMATRGWNKFAGIFIGNCITGISIAWWKWTHNAHHIACNSLDYDPDLQHLPMLAVSSKLFNSITSVFYGRQLTFDPLARFFVSYQHYLYYPIMCVARVNLYLQTILLLISKRKIPDRGLNILGTLIFWTWFPLLVSRLPNWPERVAFVLVSFCVTGIQHIQFTLNHFSGDVYVGPPKGDNWFEKQTRGTIDIACSSWMDWFFGGLQFQLEHHLFPRLPRCHLRSISPICRELCKKYNLPYVSLSFYDANVTTLKTLRTAALQARDLTNPAPQNLAWEAFNTHG

SEQ ID NO: 12 - protein, 359 aa, Synechocystis sp.
MLTAERIKFTQKRGFRRVLNQRVDAYFAEHGLTQRDNPSMYLKTLIIVLWLFSAWAFVLFAPVIFPVRLLGCMVLAIALAAFSFNVGHDANHNAYSSNPHINRVLGMTYDFVGLSSFLWRYRHNYLHHTYTNILGHDVEIHGDGAVRMSPEQEHVGIYRFQQFYIWGLYLFIPFYWFLYDVYLVLNKGKYHDHKIPPFQPLELASLLGIKLLWLGYVFGLPLALGFSIPEVLIGASVTYMTYGIVVCTIFMLAHVLESTEFLTPDGESGAIDDEWAICQIRTTANFATNNPFWNWFCGGLNHQVTHHLFPNICHIHYPQLENIIKDVCQEFGVEYKVYPTFKAAIASNYRWLEAMGKAS

SEQ ID NO: 13 - protein, 448 aa, Borago officinalis
MAAQIKKYITSDELKNHDKPGDLWISIQGKAYDVSDWVKDHPGGSFPLKSLAGQEVTDAFVAFHPASTWKNLDKFFTGYYLKDYSVSEVSKDYRKLVFEFSKMGLYDKKGHIMFATLCFIAMLFAMSVYGVLFCEGVLVHLFSGCLMGFLWIQSGWIGHDAGHYMVVSDSRLNKFMGIFAANCLSGISIGWWKWNHNAHHIACNSLEYDPDLQYIPFLVVSSKFFGSLTSHFYEKRLTFDSLSRFFVSYQHWTFYPIMCAARLNMYVQSLIMLLTKRNVSYRAHELLGCLVFSIWYPLLVSCLPNWGERIMFVIASLSVTGMQQVQFSLNHFSSSVYVGKPKGNNWFEKQTDGTLDISCPPWMDWFHGGLQFQIEHHLFPKMPRCNLRKISPYVIELCKKHNLPYNYASFSKANEMTLRTLRNTALQARDITKPLPKNLVWEALHTHG

SEQ ID NO: 14 - protein, 446 aa, Morierella alpina; identical to SEQ ID NO: 3

SEQ ID NO: 15 - protein, 4 aa, Artificial Sequence
  cytochrome b5 domain motif
HPGG

SEQ ID NO: 16 - DNA, 20 bp, Artificial Sequence
  PCR primer
atggtattacgagagcaaga

SEQ ID NO: 17 - DNA, 20 bp, Artificial Sequence
  PCR primer
tctgggatctctggttcttg

SEQ ID NO: 18 - DNA, 34 bp, Artificial Sequence
  PCR primer
gcgaagcttaaaatggtattacgagagcaagagc

SEQ ID NO: 19 - DNA, 33 bp, Artificial Sequence
  PCR primer
gcgggatccaatctaggcaatctttttagtcaa

SEQ ID NO: 20 - protein, 443 aa, C. elegans
MVVDKNASGLRMKVDGKWLYLSEELVKKHPGGAVIEQYKNSDATHIFHAFHEGSSQAYKQLDLLKKHGEHDEFLEKQLEKRLDKVDINVSAYDVSVAQEKKMVESFEKLRQKLHDDGLMKANETYFLFKAISTLSIMAFAFYLQYLGWYITSACLLALAWQQFGWLTHEFCHQQPFKNRPLNDTISLFFGNFLQGFSRDWWKDKHNTHHAATNVIDHDGDIDLAPLFAFIPGDLCKYKASFEKAILKIVPYQHLYFTAMLPMLRFSWTGQSVQWVFKENQMEYKVYQRNAFWEQATIVGHWAWVFYQLFLLPTWPLRVAYFIISQMGGGLLIAHVVTFNHNSVDKYPANSRILNNFAALQILTTRNMTPSPFIDWLWGGLNYQIEHHLFPTMPRCNLNACVKYVKEWCKENNLPYLVDDYFDGYAMNLQQLKNMAEHIQAKAA

SEQ ID NO: 21 - protein, 5 aa, Artificial Sequence
  misc_feature (1)...(5): Xaa = any amino acid
QXXHH

SEQ ID NO: 22 - protein, 5 aa, Artificial Sequence
  cytochrome b5-like heme-binding domain motif
EHPGG

SEQ ID NO: 23 - protein, 5 aa, Artificial Sequence
  misc_feature (1)...(5): Xaa = any amino acid
HXXHH